SEQUENCE LISTING (14 sequences)

SEQ ID NO: 1 - DNA, 20 bp, Artificial Sequence
  Synthetic
cactcgactggcctttcatt

SEQ ID NO: 2 - DNA, 20 bp, Artificial Sequence
  Synthetic
cggccaacgcaccaaatgaa

SEQ ID NO: 3 - DNA, 20 bp, Artificial Sequence
  Synthetic
tgtcttcccaagccggacat

SEQ ID NO: 4 - DNA, 20 bp, Artificial Sequence
  Synthetic
aggtcccccgatccgagtca

SEQ ID NO: 5 - DNA, 20 bp, Artificial Sequence
  Synthetic
gcacgtcgcaatcccgtcca

SEQ ID NO: 6 - DNA, 20 bp, Artificial Sequence
  Synthetic
cgggattgcgacgtgcgcac

SEQ ID NO: 7 - DNA, 20 bp, Artificial Sequence
  Synthetic
ccccccaaccaggattgtac

SEQ ID NO: 8 - DNA, 20 bp, Artificial Sequence
  Synthetic
tttggtagtattcgtcccat

SEQ ID NO: 9 - DNA, 20 bp, Artificial Sequence
  Synthetic
cccgttcgttttaataccgg

SEQ ID NO: 10 - DNA, 20 bp, Artificial Sequence
  Synthetic
gggagctctctggctaacta

SEQ ID NO: 11 - DNA, 20 bp, Artificial Sequence
  Synthetic
acactgactaaaagggtctg

SEQ ID NO: 12 - DNA, 20 bp, Artificial Sequence
  Synthetic
gtgtggaaaatctctagcag

SEQ ID NO: 13 - DNA, 20 bp, Artificial Sequence
  Synthetic
tctagcagtggcgcccgaac

SEQ ID NO: 14 - DNA, 20 bp, Artificial Sequence
  Synthetic
tcgctttcaagtccctgttc

(19) United States
(12) Patent Application Publication
(10) Pub. No.: US 2024/0271112 A1
(43) Pub. Date: Aug. 15, 2024

(54) CAS9 NICKASE-MEDIATED GENE EDITING

(71) Applicant: UNIVERSITY OF MASSACHUSETTS, Boston, MA (US)

(72) Inventors: Scot Andrew Wolfe, Winchester, MA (US); Ester Mintzer, Cherry Valley, MA (US); Jeremy Luban, Shrewsbury, MA (US); Jason Shohet, Shrewsbury, MA (US)

(21) Appl. No.: 18/566,367

(22) PCT Filed: May 31, 2022
(86) PCT No.: PCT/US2022/031528

§ 371 Date: Dec. 1, 2023

Related U.S. Application Data
(60) Provisional application No. 63/195,361, filed on Jun. 1, 2021.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

The present invention utilizes a Cas9 nickase which nicks a flanking target sequence to a duplicated gene sequence (e.g., a retroviral LTR). This nicking causes a genomic collapse of the sequence between the nick and the LTR, thereby deleting the sequence from the genome. Because the nickase does not introduce mutations at the target site, this method can be repeated maximize the efficiency (e.g., 100% of retroviral genome excision. For example, this method is useful to delete all PERVs within a pig genome intended for human transplantation. Further, such PERV-free cells can then be used to clone PERV-free pigs. Furthermore, this method is useful to remove amplified gene repeats in cancer cells.

### STATEMENT OF GOVERNMENTAL SUPPORT

This invention was made with government support under 5R01AI117839 awarded by the National Institutes Of Health. The government has certain rights in the invention.

### FIELD OF THE INVENTION

The present invention is related to the field of gene editing. In particular, the invention utilizes individual or paired Cas9 nickases in the proximity of repeated genomic elements (amplified genomic regions or retroviral elements) to destabilize the repeated elements, which can collapse these elements to a smaller number and remove any intervening sequences from the genome. For example, this nickase-mediated repeat removal can occur as a result of genomic deletion between two or more of these repeat elements concurrent with the loss of one repeat. Clinical applications of this method include, but are not limited to, removal of endogenous retroviral genes, removal of HIV-1 provirus and destabilization of cancer-associated amplified genomic regions resulting in selective killing of tumor cells. Consequently, the method can be used to prepare retroviral-free non-human tissues for human transplantation and for the treatment of cancer.

### BACKGROUND

CRISPR-Cas9-based genome editing systems have revolutionized genome editing approaches and are now being leveraged for a broad range of commercial and therapeutic applications. The majority of gene editing approaches focus on the utilization of double-strand breaks (DSBs) generated by a single nuclease or by a pair of nickases (Cas9 nucleases that have been modified such that one of the two catalytic centers (e.g., HNH or RuvC) is inactivated, which allows only a single strand of the DNA to be cleaved). Jinek et al., “A programmable dual-RNA-guided DNA endonuclease in adaptive bacterial immunity. Science 337:816-821 (2012); and Tsai et al., “Defining and improving the genome-wide specificities of CRISPR-Cas9 nucleases” Nat Rev Genet 17:300-312 (2016).

DSBs within eukaryotic genomes are potentially repaired by a number of different DNA-damage response pathways such as canonical non-homologous end joining (cNHEJ), homologous recombination (HR), and alternate non-homologous end joining (aNHEJ). McVey et al., “MMEJ repair of double-strand breaks (director's cut): deleted sequences and alternative endings” Trends in Genetics 24:529-538 (2008). In many instances, these repair pathways are in competition with one another (e.g. aNHEJ and HR), such that there are a mixture of different repair products that are produced within a population of nuclease-treated cells. Repair outcomes are not uniform within the population or across cell types and nuclease platforms.

What is needed are compositions and methods to selectively remove amplified genetic repeats from a genome without inducing double stranded DNA breaks or mutations.

### SUMMARY OF THE INVENTION

The present invention is related to the field of gene editing. In particular, the invention utilizes a single or combination of Cas9 nickases in the proximity of a locally repeated genomic element (e.g. amplified genomic regions or retroviral elements) to destabilize the repeated elements, which can collapse these elements to a smaller number and remove any intervening sequences from the genome. For example, this nickase-mediated repeat removal can occur as a result of genomic deletion between two or more of these repeat elements concurrent with the loss of one repeat. Clinical applications of this method include, but are not limited to, removal of endogenous retroviral genes, removal of HIV-1 provirus and destabilization of cancer-associated amplified genomic regions resulting in selective killing of tumor cells. Consequently, the method can be used to prepare retroviral-free non-human tissues for human transplantation and for the treatment of cancer.

In one embodiment, the present invention contemplates a method, comprising: a) providing; i) a Cas9 nickase protein; ii) a deoxyribonucleic acid (DNA) comprising a plurality of repeat regions; and iii) a guide ribonucleic acid (gRNA) having a sequence complementary to a target site proximate to, or overlapping with, at least one of said plurality of repeat regions; b) complexing said Cas9 nickase with said gRNA; c) engagement of the Cas9 nickase gRNA complex through Watson-Crick pairing to said target site such that said Cas9 nickase creates a nick proximate to at least one of said plurality of repeat regions; and d) deleting said at least one of said plurality of repeat regions from said DNA. In a further embodiment, the method comprises a) providing; i) a Cas9 nickase protein; ii) a deoxyribonucleic acid (DNA) comprising a plurality of repeat regions; and iii) a guide ribonucleic acid (gRNA) having a sequence complementary to a target site proximate to, or overlapping with, at least one of said plurality of repeat regions; b) complexing said Cas9 nickase with said gRNA; c) hybridizing said gRNA to said target site such that said Cas9 nickase creates a nick proximate to, or overlapping with, at least one of said plurality of repeat regions; and d) deleting said at least one of said plurality of repeat regions from said DNA. In one embodiment, the method further comprises repeating steps (b) through (d). In one embodiment, wherein the deleting comprises a genomic collapse. In one embodiment, wherein said DNA comprises at least one retrovirus, exemplified by, but not limited to, an edogenous retrovirus. In one embodiment, wherein said at least one endogenous retrovirus is a porcine retrovirus, exemplified by, but not limited to a porcine endogenous retrovirus. In one embodiment, wherein said at least one retrovirus is a human retrovirus, exemplified by, but not limited to a pathogenic human retrovirus and by a human endogenous retrovirus. In one embodiment, wherein said at least one endogenous retrovirus is HIV-1. In one embodiment, wherein said DNA is a genomic DNA. In one embodiment, the genomic DNA is an amplified genomic region from a cancer cell. In one embodiment, the cancer cell is a human cancer cell. In one embodiment, the genomic deoxyribonucleic acid is from a T-cell. In one embodiment, the T-cell is a human T cell. In one embodiment, the genomic DNA is a human DNA. In one embodiment, the genomic DNA is a porcine genomic DNA. In one embodiment, wherein the method does not induce a mutation in said DNA at the target site.

In one embodiment, the present invention contemplates a method, comprising: a) providing; i) a Cas9 nickase protein; ii) a porcine cell comprising a deoxyribonucleic acid (DNA) harboring endogenous retroviral genomes; and iii) a guide ribonucleic acid (gRNA) having a sequence complementary to a target site proximate to, or overlapping with, at least one of said endogenous retroviral long-terminal repeats; b) administering the Cas9 nickase and gRNA to the porcine cell such that said Cas9 nickase creates a nick proximate to at least one of the endogenous retroviral long-terminal repeats; and c) deleting the intervening retroviral genome between the long-terminal repeats from said porcine DNA. In a further embodiment, the invention provides a method, comprising: a) providing: i) a Cas9 nickase protein; ii) a porcine cell comprising a deoxyribonucleic acid (DNA) having a plurality of endogenous retroviral repeats; and iii) a guide ribonucleic acid (gRNA) having a sequence complementary to a target site proximate to, or overlapping with, at least one of said plurality of endogenous retroviral repeats; b) administering said Cas9 nickase and said gRNA to said porcine cell such that said Cas9 nickase creates a nick proximate to, or overlapping with, at least one of said plurality of endogenous retroviral repeats; and c) deleting said at least one of said plurality of endogenous retroviral repeats from said porcine DNA. In one embodiment, the method further comprises repeating steps (b) and (c) thereby creating an porcine cell without functional endogenous retroviruses. In one embodiment, wherein the method does not induce a mutation in said porcine DNA at the target site. In one embodiment, the deleting step c) is targeted to repeats that are associated with the termini of the endogenous retroviral element. In a preferred embodiment, there is no deletion between different retroviruses within the genome.

In one embodiment, the present invention contemplates a method, comprising: a) providing; i) a Cas9 nickase protein; ii) a patient comprising a cancer cell having a deoxyribonucleic acid (DNA) with a plurality of amplified genomic regions; and iii) a guide ribonucleic acid (gRNA) having a sequence complementary to a target site proximate to, or overlapping with, at least one of said plurality of amplified genomic regions; b) administering the Cas9 nickase and gRNA to the patient such that said Cas9 nickase creates a nick proximate to, or overlapping with, at least one of said plurality of amplified genomic regions; and c) deleting or destabilizing said at least one of said plurality of amplified genomic regions from said patient DNA. In one embodiment, the method further comprises repeating steps (b) and (c) thereby selectively killing the cancer cell. In one embodiment, wherein the method does not induce a mutation in a non-cancer cell DNA of said patient. In one embodiment, the patient is a human patient. In one embodiment, said method is not toxic to a non-cancer cell of said patient. In one embodiment, said plurality of amplified genomic regions is exemplified by more than one amplified genomic region and/or multiple copies of only one amplified region. In one embodiment, said Cas9 nickase creates a nick overlapping with at least one of said plurality of amplified genomic regions. This embodiment in preferred in cancer associated amplified genomic regions (compared to retroviral elements). In one embodiment, said method selectively kills the cancer cells. In a further embodiment, said method does not comprise repeating steps (b) and (c) and said method selectively kills the cancer cells.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9” Science 346(6213):1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and spacer RNA may be combined into a “single-guide RNA” (sgRNA) molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337(6096):816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10(11):1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32(3):279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or “linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

The term “sample” or “biopsy” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “transfection” or “transfected” refers to the introduction of foreign DNA into a cell.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Retroviruses are a class of viruses that copy its RNA genome using an encoded reverse transcriptase into DNA before inserting its genome into the host cell. Retroviral genomes once integrated in the host genome can produce new functional viral particles through transcription of its integrated DNA. Retroviral genomes can be acquired through infection of the host with a pathogenic virus (e.g. HIV-1, HIV-2 or HTLV) or can be present as a resident “endogenous” retrovirus (ERV). There are many classes of human endogenous retroviruses (HERVs) such as HERV-H and HERV-FC1.

Somatic copy number alterations (SCNA) are associated with cancer progression. SCNAs can take the form of amplification of genomic regions. For certain types of cancer, there are recurrent amplifications (amplification of a similar region of the genome in different patients), which may correspond to genes that promote cancer progression in a specific cancer type, such as MYCN in neuroblastoma or EGFR in glioblastoma. These amplifications may take the form of ten to more than one thousand copies of a specific genomic region, which can be in the form of local amplified regions or extrachromosomal circular DNAs.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents a representative illustration of a SpyCas9-sgRNA nickase targeting a single site just inside a long terminal repeat (LTR) that yields collapse of a human immunodeficiency virus (HIV) genome to a single LTR, thereby deleting the HIV genome.

FIG. 2 presents a representative illustration of a traditional nuclease-based approach for removal of the HIV-1 genome. SpyCas9-sgRNA complexes targeting the LTR generate two DSBs, where in some fraction of the genomes, the intervening sequence will be lost. Insertions or deletions (InDels) are present at the target sequence in the remaining LTR. The majority of the products that are produced are simply mutations in the LTRs at the Cas9 target site without excision of the HIV-1 genome.

FIG. 3 presents exemplary data of a PCR analysis showing the relative rates of deletion from an HIV genome using SpyCas9 nuclease (black numbers) or SpyCas9 nickase (red numbers) targeting various positions (indicated by arrows) within, and neighboring, the 5′ LTR. A 700-bp PCR product is generated by primers that overlap the boundary between the LTRs and the endogenous genomic sequence in J-Lat A1 cells, that is specific to a deletion product in this size range. For the nickase, the strand that is cleaved is indicated by the position of the arrow above or below the DNA. Target sites 628, 645 and 675 only cut near the 5′ LTR and are not present in the 3′ LTR. Thus, they yield single nicks.

FIG. 4 presents exemplary data showing GFP expression following PMA stimulation in nuclease- and nickase-treated J-Lat A1 cells. Editing by the SpyCas9 nuclease targeting the T5 site and the nickase targeting the 628 site provide similar levels of reduction of GFP expression. Arrows indicate editing suppression of GFP expression. +PMA indicates J-Lat A1 cells treated with PMA following editing. Biological triplicate where error bars indicated+/−s.e.m.

FIG. 5 presents exemplary data showing that SpyCas9 nuclease mutates its target sequence at a high rate in the treated HIV genome (small insertions and deletions that disrupt the target sequence), whereas SpyCas9 D10A nickase (nSpyCas9) does not produce appreciable mutations. Mutation rate determined by TIDE analysis of PCR amplicons spanning the target sites in full length 5′ LTR target sequence of J-Lat A1 cells following treatment with SpyCas9 nuclease or nickase.

FIG. 6 presents exemplary data showing that SpyCas9 nickase achieves increased HIV genome deletion rates with serial treatment. J-Lat A1 cells were electroporated with Cas9 nuclease or nickase protein-sgRNA complexes targeting the 628 site and then recovered for multiple days. This process was repeated five times. J-Lat A1 genomes were harvested from each treatment group and then subjected to qRT-PCR (SYBR Green) using a primer set that is specific for amplification of the collapsed LTR sequence. Technical triplicate, error bars indicate s.e.m.

FIG. 7 presents exemplary data showing an analysis of HIV-1 genome size in J-Lat A1 clones isolated from a population treated with five rounds of SpyCas9 nickase at site 616 or 628 that generate single nicks abutting the 5′ LTR. Blue arrow indicates the expected full length genome size and the red arrow indicates the single LTR size after amplification with primers flanking the integration site. Seven of the fifteen clones (red numbers) contain a single LTR.

FIG. 8 presents exemplary data showing an analysis of HIV-1 genome excision rate in the J-Lat 10.6 population treated for 1 or 2 rounds with SpyCas9 nuclease (WT) or SpyCas9 nickase (D10A) RNPs via electroporation at the T5, 616 or 616/3812 sites. The red arrow indicates the single LTR amplicon size, which appears to be increasing in intensity between the first and second rounds for the nickase-treated samples.

FIG. 9 presents exemplary data showing an analysis of HERV-FC1 excision rate in Jurkat cells treated with SpyCas9 nuclease (WT) or SpyCas9 nickase (D10A) RNPs via electroporation at the indicated target site(s). Numbers indicate the position within the FC1 genome of the target sequence, where red numbers are neighboring the 5′ LTR and blue numbers are neighboring the 3′ LTR. The red arrow indicates a PCR amplicon using primers flanking the HERV-FC1 genome that is consistent with the presence of a single LTR.

FIG. 10 presents exemplary data showing an analysis of HERV-FC1 deletion in primary T-cells from two donors treated with either SpyCas9 nuclease (WT) or nickase (D10A) RNPs via electroporation. A single guide targeting the PBS (358) was used. The PCR amplicon using primers flanking the HERV-FC1 genome is consistent with the presence of a single LTR.

FIG. 11 presents exemplary data showing an analysis of PERV deletion in porcine PK-15 cells treated with SpyCas9 nuclease (WT) or a Cas9 nickase (D10A) RNPs via electroporation with guides 6-1 (targeting a single PERV) or 6-2 (targeting 18 PERVs) and then recovered for multiple days. This process was repeated five times. PK15 genomes were harvested from each treatment group and then subjected PCR amplification using primers specific for one targeted PERV. The red arrow indicates a PCR amplicon using primers flanking the PERV genome that is consistent with the presence of a single LTR (deletion of the intervening retroviral genome).

FIG. 12 presents exemplary data showing the toxicity of a SpyCas9 nuclease or a Cas9 D10A nickase when programmed with an sgRNA complementary to 179 HERV-H elements in Kelly cells.

FIG. 12A: Celigo proliferation analysis of Kelly cells treated with SpyCas9 D10A nickase RNP targeting HERV-H or HERV-FC1. Targeting HERV-FC1, which is a single copy per genome, is not toxic whereas targeting HERV-H, which is present in >1000 copies, is toxic to the cells. Individual points represent the mean of 6 technical replicates. Error bars indicate s.e.m.

FIG. 12B: Analysis of Annexin V and PI staining of Kelly cells treated with SpyCas9 D10A nickase or SpyCas9 nuclease RNP targeting HERV-H or HERV-FC1 at 96 hours post treatment. Both the nuclease and nickase targeting HERV-H initiate cell death. Mock=cells electroporated without Cas9.

FIG. 13 presents an exemplary FISH analysis of MycN genomic copies (green) relative to a D2Z1 control locus (red) in two neuroblastoma cell lines.

FIG. 13A: SYSY cells in which MycN is not amplified

FIG. 13B: Kelly cells in which MycN is highly amplified

FIG. 14 presents a representative schematic of MycN targeting sgRNA positions within and outside the gene. Rectangles indicate the primary exons of MycN and AAAAA denotes the polyA tail. Arrows denote the approximate position of the target sites for each guide. Positions of the arrows above or below the sequence indicate the strand that is cleaved when using SpyCas9 D10A nickase.

FIG. 15 presents exemplary data of a Celigo analysis of cell growth of neuroblastoma cell lines following treatment with SpyCas9 nuclease RNPs programmed to target different elements within the genome.

FIG. 15A: SY5Y cells treated with SpyCas9 nuclease targeting HERV-FC1 (FC1) or two regions within MycN (position 69 or 1262). No appreciable impact on cell growth is observed.

FIG. 15B: Kelly cells treated with SpyCas9 nuclease targeting HERV-FC1 (FC1) or two regions within MycN (position 69 or 1262). MYCN targeting nuclease is toxic to these cells with a gene amplification. Mock=cells electroporated without Cas9. Individual points represent the mean of 6 technical replicates. Error bars indicate s.e.m.

FIG. 16 presents exemplary data of a Celigo analysis of cell growth of neuroblastoma cell lines following treatment with SpyCas9 D10A nickase RNPs programmed to target different elements within the genome.

FIG. 16A: SYSY cells treated with SpyCas9 D10A nickase targeting HERV-FC1 (FC1) or two regions within MycN (position 69 or 1262). No appreciable impact on cell growth is observed.

FIG. 16B: Kelly cells treated with SpyCas9 D10A nickase targeting HERV-FC1 (FC1) or two regions within MycN (position 69 or 1262). MYCN targeting nuclease is toxic to these cells with a gene amplification. Mock=cells electroporated without Cas9. Individual points represent the mean of 6 technical replicates. Error bars indicate s.e.m.

FIG. 17 presents exemplary data of genome editing rates for SpyCas9 nuclease programmed with MycN guides delivered as RNPs by electroporation to SYSY cells. Editing rates were determined by TIDE analysis of Sanger sequencing from PCR amplicons spanning the target region from the treated cell population.

FIG. 18 presents exemplary data of a Celigo analysis of cell growth of neuroblastoma cell lines following treatment with SpyCas9 nuclease or SpyCas9 D10A nickase RNPs programmed to target four different elements within the MycN locus (position 69, 73, 116 or 409; See, FIG. 14) or HERV-FC1 (FC1). Mock=cells electroporated without Cas9. Individual points represent the mean of 6 technical replicates. Error bars indicate s.e.m..

FIG. 18A: SYSY cells treated with SpyCas9 nuclease.

FIG. 18B: Kelly cells treated with SpyCas9 nuclease.

FIG. 18C: SYSY cells treated with SpyCas9 D10A nickase.

FIG. 18D: Kelly cells treated with SpyCas9 D10A nickase.

FIG. 19 presents exemplary data of quantification of the relative deletion rate of the HIV proviral genome in J-Lat A1 cells based on ImageJ quantification of the bands in the gel image in FIG. 3. Numbers in the legend indicate the position of the gRNA targeting the Cas9 nuclease or Cas9 D10A nickase. Target sites 628, 645 and 675 only cut near the 5′ LTR and are not present in the 3′ LTR. Thus, they yield single nicks. The Cas9 nickase programmed with gRNA 628 has similar deletion activity to Cas9 nuclease targeting throughout the LTR.

FIG. 20 presents exemplary data for genomic deletion of the HIV genome from JLat A1 cells.

FIG. 20A—schematic of the HIV genome present in JLat A1 cells, where many of the HIV genes have been removed and replaced by a GFP expression cassette.

FIG. 20B—PCR analysis showing the relative rates of deletion from an HIV genome using SpyCas9 D10A nickase targeting a single site (“single nickase”) or targeting two sites one inside each LTR (“nick inside each LTR”), where the position of the guide within the genome is indicated by the number and the strand that is cleaved is indicated by the position of the arrow above or below the DNA. A 700-bp PCR product is generated by primers that overlap the boundary between the LTRs and the endogenous genomic sequence in J-Lat A1 cells, that is specific to a deletion product in this size range (indicated by the magenta arrow).

FIG. 20C—quantification of the relative deletion rate of the HIV proviral genome in J-Lat A1 cells based on ImageJ quantification of the bands in the gel image in FIG. 20B.

FIG. 21 presents exemplary data showing that SpyCas9 nickase limits the amount of mutagenesis at its target site. in JLat A1 cells following each round of serial treatment by electroporated with Cas9 nuclease or Cas9 D10A nickase protein-sgRNA complexes targeting the 628 site and then recovered for multiple days. This process was repeated five times. (Genome deletion data shown in FIG. 6) J-Lat A1 genomes were harvested from each treatment group and then subjected to PCR amplification using a primer set that is specific for amplification of the 5′ LTR sequence spanning the 628 target site (which would be lost if the genome had collapsed). Illumina sequencing was performed on these PCR amplicons and the mutagenesis rate (InDel %) was determined.

FIG. 22 presents exemplary UDiTaS data estimating the collapse rate of the HERV-FC1 locus after treatment of Jurkat cells by electroporation with Cas9 nuclease (DSB) or Cas9 D10A nickase protein complexed with one or two guide RNAs (target position indicated by the numbers). Genomic DNA from the treatment groups was subjected to UDiTaS library preparation followed by Illumina sequencing. Dots represent three biological replicates.

FIG. 23 presents exemplary data showing an analysis of HERV-FC1 genome size in Jurkat clones isolated from a population treated with five rounds of SpyCas9 nickase at site 358 and 7558 that generate single nicks abutting the 5′ and 3′ LTR. 21 of the 23 clones contain a single LTR.

FIG. 24 presents exemplary data showing the impact of siRNA knockdown of various DNA repair factors on the deletion rate of HERV-FC1 driven by SpyCas9 nickase targeting site 358. Cells were treated with each siRNA 48 hours prior to electroporation with the SpyCas9 nickase protein RNA complex (RNP). HERV-FC1 deletion rates at 3 days following treatment were determined by qPCR relative to the control (no siRNA) sample. NT=non-target siRNA. Knockdown of Rad51 increases the deletion rate ˜3 fold. Technical triplicate experiment.

FIG. 25 presents exemplary data showing the impact of thymidine block of cell cycle progression on the deletion rate of HERV-FC1 driven by SpyCas9 nuclease or SpyCas9 nickase targeting site 358. Cells were treated with 2 mM Thymidine for 18 hours prior to electroporation with the SpyCas9 nickase protein RNA complex (RNP). HERV-FC1 deletion rates at 3 days following treatment were determined by PCR relative to the control (no thymidine treatment). DSB=treatment with Cas9 nuclease; Nick=treatment with Cas9 D10A nickase. Thymidine treatment reduced the HERV-FC1 deletion rate for both the nuclease and nickase.

FIG. 26 presents exemplary data showing MycN amplification toxicity by comparing the fraction of Kelly cells containing MycN amplification that are in each phase of the cell cycle. Kelly cells were nucleofected with the indicated Cas9 nuclease (WT) RNP or D10ACas9 nickase (D10A) programmed with the indicated guideRNA(s). Cas9 Nuclease (WT) or D10A nickase (D10A) RNP composition in each sample is indicated on the X-axis. Camptothecin treatment is included to provide a reference for non-specific replication fork collapse and DSB introduction.

FIG. 27 presents exemplary data showing MycN amplification toxicity by comparing viability and the fraction of Kelly cells containing an MycN amplification that were undergoing apoptosis. Kelly cells were nucleofected with the indicated Cas9 nuclease (WT) RNP or D10ACas9 nickase (D10A) programmed with the indicated guideRNA(s). Cas9 Nuclease (WT) or D10A nickase (D10A) RNP composition in each sample is indicated on the X-axis. Camptothecin treatment is included to provide a reference for non-specific replication fork collapse and DSB introduction.

### DETAILED DESCRIPTION OF THE INVENTION

The present invention is related to the field of gene editing. In particular, the invention utilizes individual or multiple Cas9 nickases in the proximity of repeated genomic elements (amplified genomic regions or retroviral elements) to destabilize the repeated elements, which can collapse these elements to a smaller number and remove any intervening sequences from the genome. In one preferred embodiment, the invention utilizes individual Cas9 nickases in the proximity of repeated genomic elements (amplified genomic regions or retroviral elements). For example, this nickase-mediated repeat removal can occur as a result of genomic deletion between two or more of these repeat elements concurrent with the loss of one repeat. Clinical applications of this method include, but are not limited to, removal of endogenous retroviral genes, removal of HIV-1 provirus and destabilization of cancer-associated amplified genomic regions resulting in selective killing of tumor cells. Consequently, the method can be used to prepare retroviral-free non-human tissues for human transplantation and for the treatment of cancer.

### I. Cas9 Nickase-Mediated Genomic Collapse

A targeted single nick within a genome (not a DSB) can drive a variety of DNA repair events in cells: in conjuction with a donor DNA-HR mediated precise repair, in conjunction with Cas9 nickase fused to a cytosine or adenosine deaminases—base transition, and in conjunction with Cas9 nickase fused to a reverse transcriptase (prime editing)—local sequence changes programmed by a RNA delivered in cis. Davis et al., “Two Distinct Pathways Support Gene Correction by Single-Stranded Donors at DNA Nicks” Cell Reports 17:1872-1881 (2016); Davis et al., “Homology-directed repair of DNA nicks via pathways distinct from canonical double-strand break repair” Proc National Acad Sci 111, (2014); Bothmer et al., “Characterization of the interplay between DNA repair and CRISPR/Cas9-induced DNA lesions at an endogenous locus” Nat Commun 8:13905 (2017): Rees et al., “Base editing: precision chemistry on the genome and transcriptome of living cells” Nat Rev Genet 70:3240 (2018). Anzalone, A. V. et al. “Search-and-replace genome editing without double-strand breaks or donor DNA.” Nature 576, 149-157 (2019).

Single nicks have been shown to be able to drive gene conversion between the beta-globin gene (HBB) and the delta-globin gene (HBD), although the precise DNA repair mechanism utilized for gene conversion has not been defined. Bothmer et al., “Characterization of the interplay between DNA repair and CRISPR/Cas9-induced DNA lesions at an endogenous locus” Nat Commun 8:13905 (2017).

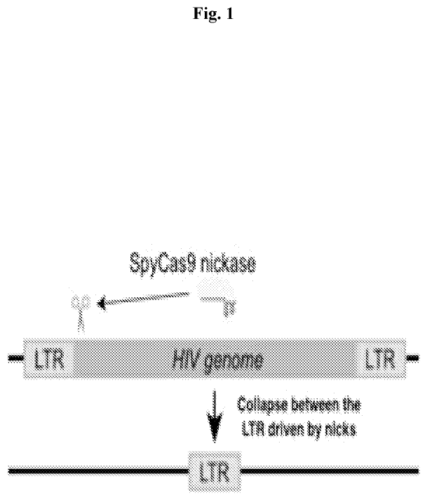

One of the advantages of a single nickase for HR-based events is that—unlike nucleases —a nickase does not create mutations at the target site at an appreciable rate, in contrast to a DSB generated by a nuclease which produces mutations at high rates. Although it is not necessary to understand the mechanism of an invention, it is believed that a nickase collapses a genomic sequence between the long terminal repeats (LTRs), which are tandem repeats that are found at each end of the genomic integrated viral genome (e.g., a provirus). See, FIG. 1.

Nickase-mediated genomic collapse is analogous, in some regards, to the above referenced nickase-mediated gene conversion in that it is harnessing proximal regions of homology to generate a change within the genome. However, it differs in an important way. Nickase-mediated genomic collapse harnesses a homology-based repair pathway to delete an intervening sequence between the elements of homology, such as gene elements within the genomic sequence. This approach leverages existing nuclease technology and endogenous cellular DNA repair pathways to achieve this goal.

The efficiency of nickase-mediated targeted collapse of sequences is modest—a few percent per treatment of the cells (described below). This nickase-based approach, because it is only moderately mutagenic at a target sequence, allows repeated treatment of a cellular population to drive the reaction toward complete repeat collapse. Nickase-mediated genomic collapse leverages existing nuclease technology and endogenous cellular DNA repair pathways. The efficiency of a nickase targeted collapse of sequences varies depending on target sequence and local homology between the repeats—from a few percent to >50% per treatment of the cells. Consequently, a nickase-mediated treatment can dramatically outperform nucleases for removal of sequences between two elements of homology (50%-80% deletion rate following five rounds of treatment).

In one embodiment, the present invention contemplates a method for genome manipulation comprising a nickase complexed with a guide RNA or RNAs that are useful for achieving specific therapeutic and/or commercial tasks. In one embodiment, guide RNA or RNAs hybridize in proximity to repeat elements, wherein a near proximity hybridization is positively correlated with an increased rate of repeat collapse. In one embodiment, the guide RNA or RNAs hybridize in near proximity to, or overlapping with, amplified cancer genes. In one embodiment, the amplified cancer genes are susceptible to repeat collapse or destabilization that promotes cellular toxicity. In one embodiment, the nickase complexed to the guide RNA or RNAs does not cause mutagenesis or genome instability in normal cells lacking the amplified genomic region of cancer cells.

### II. Endogenous Retroviral Removal

In one embodiment, the present invention contemplates a method comprising a Cas9 nickase mediated removal of endogenous retroviruses (ERVs) from genomes. For example, there has been a long felt need to eliminate porcine ERVs (PERVs), which are a barrier to the utilization of pig organs in xenotransplantation.

There is an unmet need for donor organs. nytimes.com/interactive/2018/11/14/magazine/tech-design-xenotransplantation.html; and fortune.com/2017/04/12/need-an-organ-transplant-this-pork-company-will-be-happy-to-oblige/. There are several companies that provide pig organs as one potential solution to meet this need; i) Egenesis; egenesisbio.com; ii) Hangzhou Qihan Bio; biocentury.com/bc-week-review/financial-news/completed-offerings/2018-07-27/chinese-xenotransplantation-company; and iii) Xenotherapeutics; xenotherapeutics.org.

Nuclease-based methods have been developed to inactivate the 40 to 80 PERVs present within a pig genome by mutating the PERV polymerase gene. Cloning-based approaches have resulted in the successful creation of pigs without functional PERVs. Niu et al., “Inactivation of porcine endogenous retrovirus in pigs using CRISPR-Cas9” Science 357:1303-1307 (2017); and Yang et al., “Genome-wide inactivation of porcine endogenous retroviruses (PERVs)’ Science 350(6264):1101-1104 (2015). However, this process is extremely inefficient and suffers from an extensive genome instability and toxicity simultaneously with the generation of large numbers of double strand breaks (DSBs) within a genome. In addition, the PERVs are not removed by mutation of the polymerase gene, just inactivated. So it is conceivable that these sequences could be reactivated by reversion mutations.

In one embodiment, the present invention contemplates a method for completely removing ERV coding sequences from a genome. Consequently, nickase-mediated genomic collapse is not susceptible to ERV reversion mutation. In addition, other genes also need to be inactivated to achieve the required immune tolerance for pig organ transplant into a human. Hryhorowicz et al., “Genetically Modified Pigs as Organ Donors for Xenotransplantation” Mol. Biotechnol. 59:435-444 (2017).

In one embodiment, the present invention contemplates a method comprising a Cas nickase for collapsing at least one pathogenic retroviral genome or one pathogenic genomic duplication. In one embodiment, the pathogenic genomic duplication resides within a MECP2 gene. Ramocki, M. B., Tavyev, Y. J. & Peters, S. U. “The MECP2 duplication syndrome.” Am. J. Med. Genet. 152A, 1079-1088 (2010). In one embodiment, the pathogenic retroviral genome resides within an HIV-1 provirus. In one embodiment, the pathogenic retroviral genome resides within a retrovirus or a lentiviruses (e.g. HTLV-1). Satou, Y. et al. “The retrovirus HTLV-1 inserts an ectopic CTCF-binding site into the human genome.” Proc Natl Acad Sci USA 113, 3054-3059 (2016) The data presented herein describes a Cas nickase-mediated repeat collapse for an efficient excision of HIV-1 provirus from the human genome and the removal of human and porcine ERVs.

### A. SpyCas9 Nickase Deletion Of HIV-1 Provirus

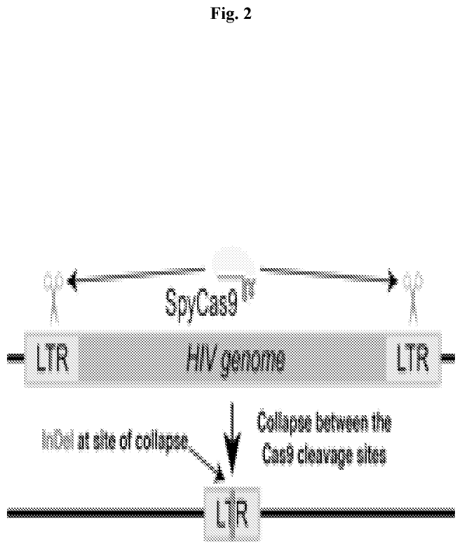

Mutation-based methods have been developed to increase the excision/removal rate of the HIV-1 genome between nucleases targeting sites that are present in the long terminal repeats (LTRs). For example, SpyCas9 target sites have been identified within the LTR that function with modest efficiency for the nuclease-based (DSB) excision of the intervening DNA sequence. See, FIG. 2.

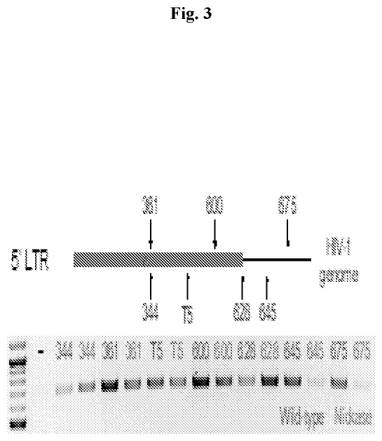

The efficiency of HIV-1 genome collapse mediated by SpyCas9 nickase (e.g., D10A; nSpyCas9) was compared to these traditional nuclease mutation-based methods, as there is literature evidence that nickases can promote gene conversion between neighboring regions of homology. The data showed that nSpyCas9 targeted to a LTR resulted in a modest rate of collapse of the HIV genome (e.g. T5 site for nuclease and nickase) in infected J-Lat A1 cells. See, FIG. 3 & Table 2.

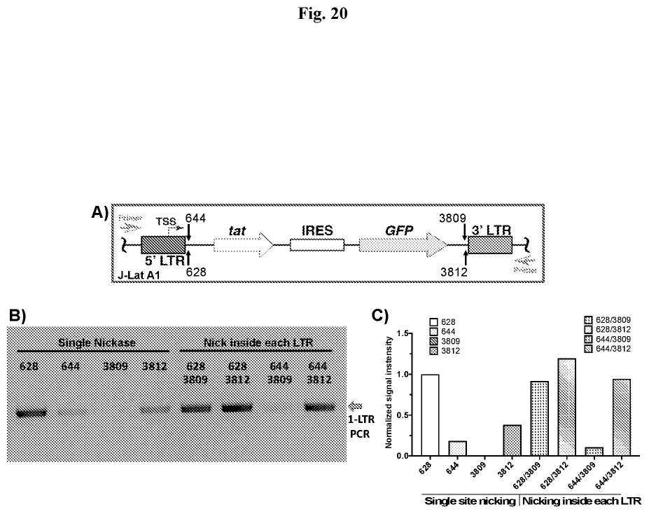

Surprisingly, it was found that SpyCas9 nickase targeting a sequence overlapping the tRNALys primer binding site (PBS, 628) contained a single defective copy of HIV-1 caused genome collapse at a rate that was just as efficient as any nuclease target site (e.g., site 600; FIGS. 3 & 19). Although it is not necessary to understand the mechanism of an invention, it is believed that there may be synergy through the generation of a single nick just inside the LTRs at both ends of the HIV-1 genome (e.g. within the PBS [guide 628] and within nef [guide 3812]; FIG. 20).

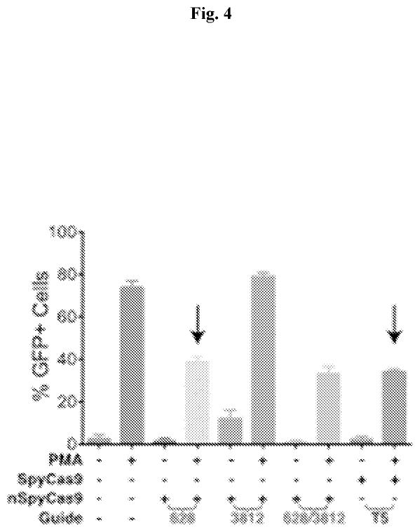

J-Lat A1 cells contain a GFP reporter in place of much of the HIV genome that is expressed upon PMA stimulation. This reporter provides an estimate of the functional HIV-1 genomes after editing. PMA stimulation of treated cells suggests that the SpyCas9 nickase targeting the 628 site is just as effective at suppressing GFP expression as SpCas9 nuclease targeting the T5 site. See, FIG. 4. Thus, single nicks produce substantial collapse of the HIV genome with functional consequences.

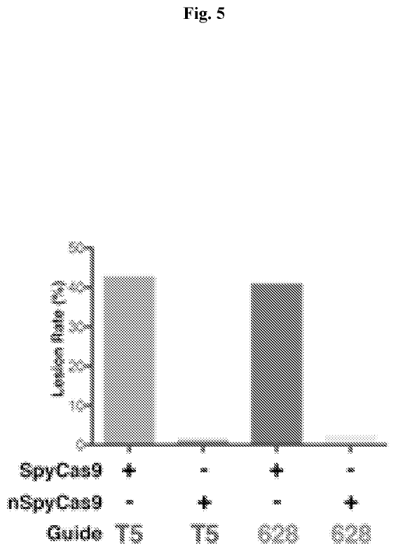

One of the challenges of targeting HIV-1 with SpyCas9 nuclease is a potential for mutations that are produced at a target site to permit “escape” by protecting the mutant genomes from further nuclease cleavage. Wang et al., “CRISPR-Cas9 Can Inhibit HIV-1 Replication but NHEJ Repair Facilitates Virus Escape” 24:522-526 (2016). Indeed, when the 5′ LTR region in SpyCas9 nuclease-treated J-Lat A1 cells is sequenced, a large fraction of the remaining full-length genomes have mutations. See, FIG. 5. However, there are negligible mutations at the target site in the SpyCas9 nickase-treated cells, which is consistent with published studies indicating that DNA nicks are repaired with high fidelity in most cells. The implications of precise repair of a nicked target site suggest successful serial treatments with a Cas9 nickase. A serial Cas9 nickase treatment would be expected to produce a progressive increase in the number of collapsed HIV genomes. In contrast, due to mutations at the target site in full-length genomes, nuclease-treated cells would most likely be recalcitrant toward serial editing.

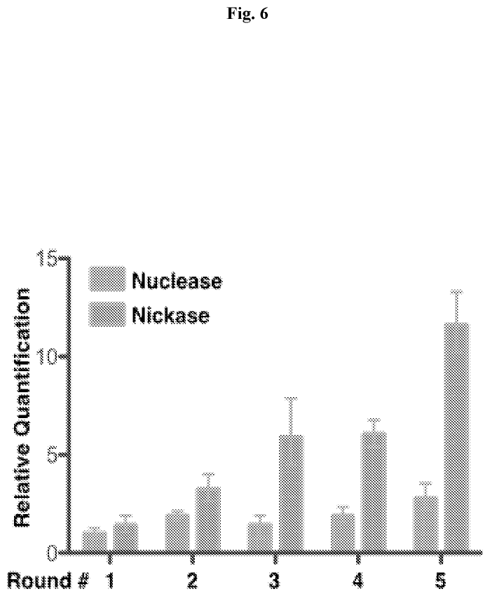

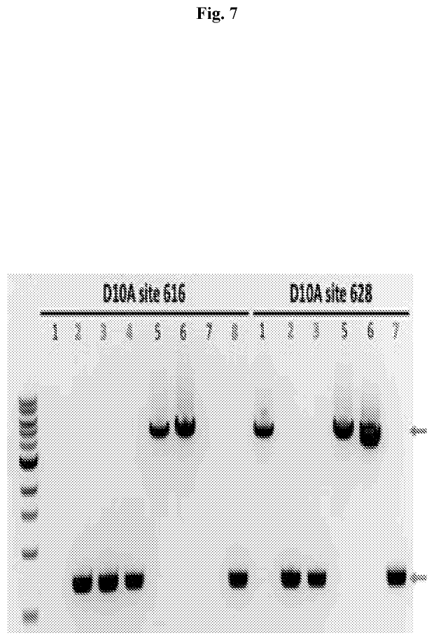

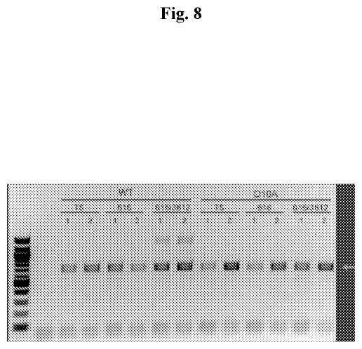

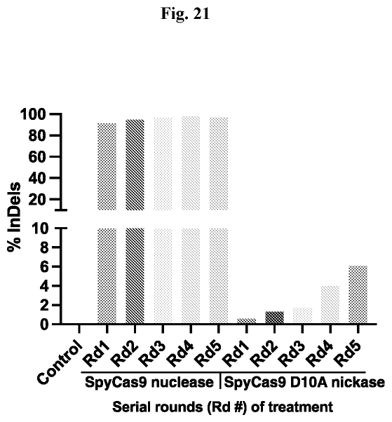

To test this hypothesis, J-Lat A1 cells were serially treated with SpyCas9 nuclease protein-sgRNA complex or SpyCas9 nickase protein-sgRNA complex by nucleofection in five (5) serial treatments and then the relative amount of collapsed HIV-1 LTRs was quantified by qRT-PCR. The data showed a progressive increase number of collapsed genomes (single LTRs) in the nickase-treated cells, while the rate of LTR collapse in the nuclease-treated sample remained relatively stagnant across the serial treatments. See, FIG. 6. Illumina sequencing of the genomic target site in the full length viral integrant reveals why repeated rounds of nuclease treatment do not increase the retroviral deletion rates. In the nuclease-treated samples >90% of the genomic target sites are mutated, so cannot be recut, whereas after five rounds of treatment by the Cas9 nickase the vast majority of target sites (>90%) are not mutated (FIG. 21). To estimate the rate of HIV-1 genome collapse in J-Lat A1 treated cells, single-cell clones were generated after five rounds of SpyCas9 nickase treatment. Analysis of these clones indicates that 7 of 15 clones achieved collapse to a single LTR with concomitant excision of the HIV-1 genome. See, FIG. 7. This serial SpyCas9 nickase treatment was successfully repeated with J-Lat 10.6 cells that contain a single full-length attenuated provirus. See, FIG. 8.

### B. SpyCas9 Nickase HERV-FC1 Deletion in A Human Genome

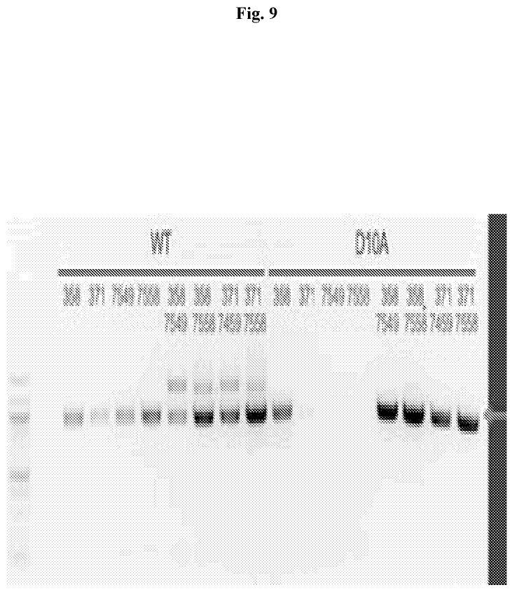

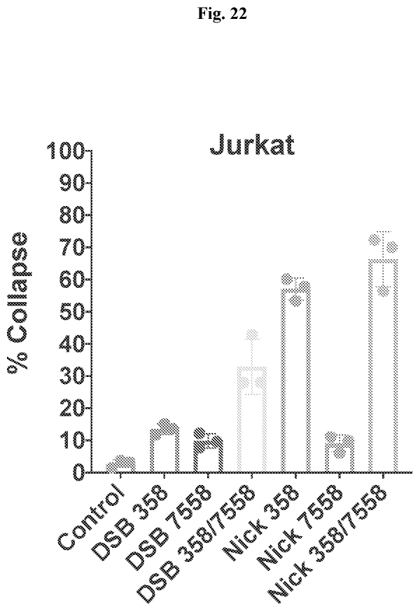

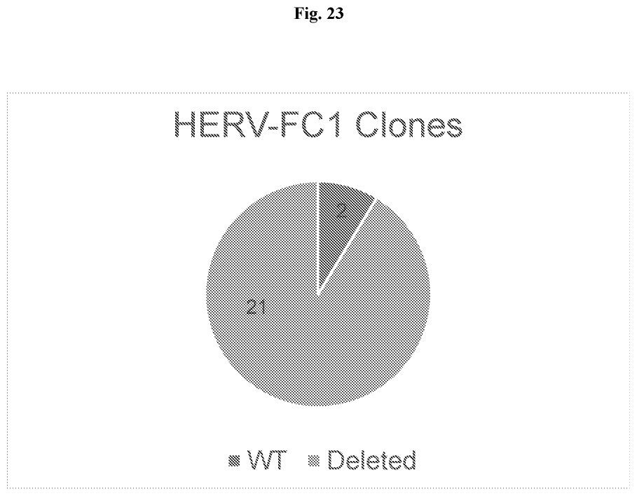

In one embodiment, the present invention contemplates a method for excising at least one HERV from a human genome. HERV-FC1 is present in a single copy on the X-chromosome. Benit et al., “Characterization of the low-copy HERV-Fc family: evidence for recent integrations in primates of elements with coding envelope genes” Elsevier 312:159-168 (2003). In a male-derived cell line with a single X chromosome (Jurkat cells), the impact of editing at a single HERV locus can be characterized. Treating HERV-FC1 with SpyCas9 nuclease or nickase just outside an LTR region caused excision from the genome in some fraction of the treated cells. See, FIG. 9. Making two nicks (one at each end of the genome just outside the LTRs) yielded even higher rates of deletion from the genome. UDiTaS analysis was performed to determine the rate of deletion of the HERV-FC1 genome in each treatment group. These data reveal that single or double nicks by a Cas9 D10A nickase can be even more effective than DSBs created at the corresponding sites by the nuclease, where ≥50% of the genomes show evidence of collapse (FIG. 22). Jurkat cells were serially treated with SpyCas9 nickase protein-sgRNA complex targeting the 358 and 7558 sites by nucleofection in five (5) serial treatments. To estimate the rate of HERV-FC1 genome collapse in Jurkat treated cells, single-cell clones were generated after five rounds of SpyCas9 nickase treatment. Analysis of these clones indicates that 21 of 23 clones achieved collapse to a single LTR with concomitant excision of the HERV-FC1 genome. See, FIG. 23 and Table 2.

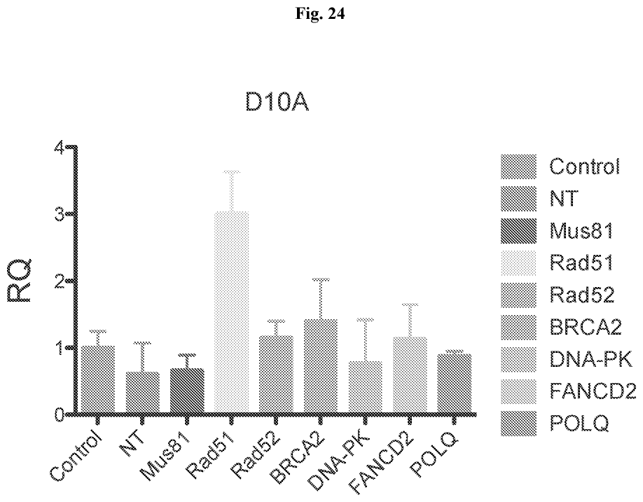

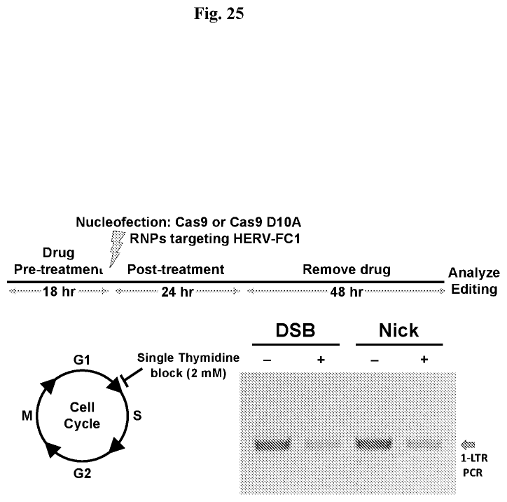

The rate of nickase mediated deletion of the HERV-FC1 is suppressed by the DNA repair factor Rad51. siRNA knockdown of Rad51 in Jurkat cells increased the rate of deletion of HERV-FC1 driven by Cas9 D10A nickase targeting site 358 (FIG. 24). Nickase-mediated collapse appears to be dependent on the progression through S-phase of the cell cycle (FIG. 25).

### C. SpyCas9 Nickase HERV-FC1 Deletion in Human Primary CD4⁺ T-Cells

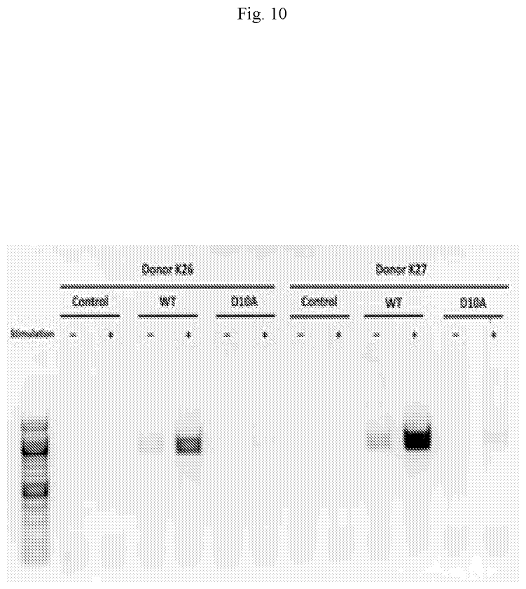

In one embodiment, the present invention contemplates a method for deleting HERV from human primary CD4⁺ T-cells. The T-cells were isolated from peripheral blood mononuclear cells (PBMCs) obtained from healthy human donors. The T-cells were either stimulated for three days after isolation with CD3/CD28 antibodies to induce proliferation or left unstimulated. HERV-FC1 deletion was greatly enhanced in stimulated cells when compared with unstimulated. See, FIG. 10. These data indicate that progression through the cell cycle plays a role in the DNA repair pathway that leads to deletion of the ERV genome. A SpyCas9 nuclease was observed to generate a higher rate of HERV-FC1 deletion than a Cas9 nickase.

### D. SpyCas9 Nickase PERV Deletion From Genomic DNA

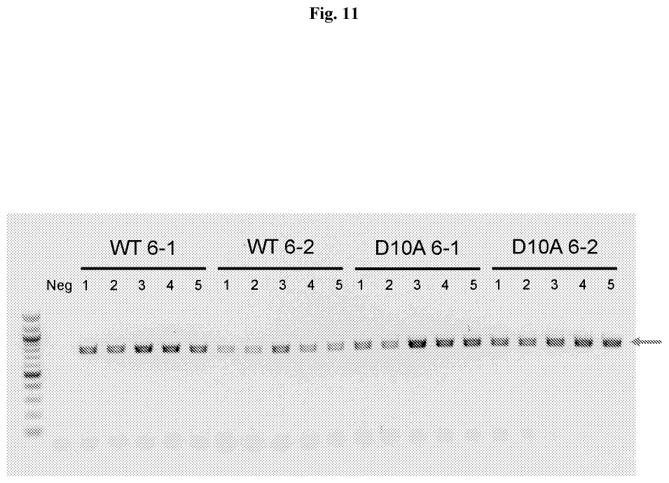

In one embodiment, the present invention contemplates deleting porcine endogenous retroviruses from a porcine genome. In one embodiment, the porcine genome is from PK-15 cells. Yang et al., “Genome-wide inactivation of porcine endogenous retroviruses (PERVs)’ Science 350(6264):1101-1104 (2015). A guide targeting a single PERV on the X-chromosome (guide 6-1) was compared with a guide targeting that same PERV plus 17 other PERV loci (guide 6-2). Both the Cas9 nuclease and Cas9 nickase were able to delete a single PERV with either guide. See, FIG. 11 & Table 1. In addition, gene editing rates of 95-98% were achieved with a single Cas9 nuclease treatment (data not shown), meaning that the target sequence is rapidly and efficiently ablated by the wild type (WT) Cas9 nuclease which would be expected to not be amenable to further disruption. Thus, additional nuclease treatments will not produce an appreciable increase in deletion rates, whereas a repeated Cas9 nickase treatment would continue to cause efficient deletion providing a method to delete all PERVs from a pig genome.

TABLE 1
Representative guide RNA positions for PERV genomic regions

| Guide | Locus | Sequence (5′-3′) | susScr11 coordinates |
|---|---|---|---|
| PERV 6-1 | PERV | CACTCGACTGGCCTTTCATT | chrX:73761201-73761220 |
| PERV6-2 | PERV | CGGCCAACGCACCAAATGAA | chrX:71402811-71402830* |

*Example locus-there are 18 cognate sites in the genome

### III. Cas9 Nickase Cancer Treatment

In one embodiment, the present invention contemplates a method comprising a pharmaceutically acceptable Cas9 nickase composition for selective toxic killing of cancer cells harboring amplified genes. In one embodiment, the present invention contemplates a method for Cas9 nickase-mediated toxicity to cancer cells containing amplified genomic regions. In one embodiment, the cell toxicity comprises repeat genomic collapse mediated by a catastrophic DNA repair outcome. In one embodiment, the cell toxicity comprises a direct gene effect mediated by the Cas9 nickase.

In one embodiment, the present invention contemplates a method comprising targeting a nick to a gene amplified region in a cancer cell that is highly toxic to the cell. In one embodiment, the highly toxic effect comprises a reduced cancer cell proliferation. Since these nicks should not be toxic to normal cells, it provides a selective method to target any cancer with a gene amplification.

### A. Cancer Cells and Amplified Genomic Repeat Regions

Given the large number of cancers that have amplified loci, this method is a universal targeting strategy to remove specific gene amplifications from the genome. Because of the existence of multiple guide RNA target sites in cancer cell gene amplifications, the impact of a target site mutation before or during therapy can be circumvented merely by targeting a different guide RNA target site within the same locus. In analogy to HIV-1 cocktail drug therapies, the present invention contemplates the administration of a plurality of (e.g., a cocktail) of Cas9 nickases, each targeted to different sites with different guide RNAs. Since Cas9 nickases have limited toxicity to normal cells and can be targeted to non-coding sequences within the genome within the amplified region of interest, the therapeutic index is high

Many types of cancers, such as neuroblastoma and glioblastoma, have gene amplifications (e.g., MycN & EGFR, respectively) that are a common form of dysregulation that drives oncogenesis. Zack et al., “Pan-cancer patterns of somatic copy number alteration” Nat Genet 45:1134-1140 (2013); and Beroukhim et al., “The landscape of somatic copy-number alteration across human cancers” Nature 463:899-905 (2010). Cells harboring gene amplifications are known to be sensitive to Cas9-induced double strand breaks that target the amplified region. Wang et al., “Identification and characterization of essential genes in the human genome” Science 350:1096-1101 (2015). However, using nucleases to target these amplified elements in cells is suboptimal, as the endogenous locus in normal cells also experience DSBs. DSBs in normal cells can lead to local deletions and\or other types of genomic rearrangements with other DSBs in a genome. Kosicki et al., “Repair of double-strand breaks induced by CRISPR-Cas9 leads to large deletions and complex rearrangements” Nat Biotechnol 36:765-771 (2018); Frock et al., “Genome-wide detection of DNA double-stranded breaks induced by engineered nucleases” Nat Biotechnol 33:179-186 (2015); and Tsai et al., “GUIDE-seq enables genome-wide profiling of off-target cleavage by CRISPR-Cas nucleases” Nat Biotechnol 33:187-197 (2015). These unwanted types of collateral genomic damage reduce the utility of Cas9 nucleases for targeting gene amplified regions in cancer if there are not unique sequences associated with these elements. If patient-specific unique sequence elements are present (e.g. in the form of translocations), the treatment requires a nuclease tailored for each patient.

### B. Cas9-Nickase Targeting of Cancer Cell Amplified Repeat Regions

The data presented herein shows the impact of targeting human endogenous retroviruses (HERVs) within the genome using a SpyCas9 D10A nickase which has an inactivated RuvC nuclease center. Jinek et al., “A programmable dual-RNA-guided DNA endonuclease in adaptive bacterial immunity” Science 337:816 821 (2012). The HERV-H family was targeted which has ˜1000 copies in the human genome. Jern et al., “Definition and variation of human endogenous retrovirus H” Virology 327:93-110 (2004). In principle, DNA nicks are not thought to be toxic, even though they have been reported to occur tens of thousands of times each day in a cell. Caldecott, K. W., “Single-strand break repair and genetic disease” Nat Rev Genet 9:619-631 (2008).

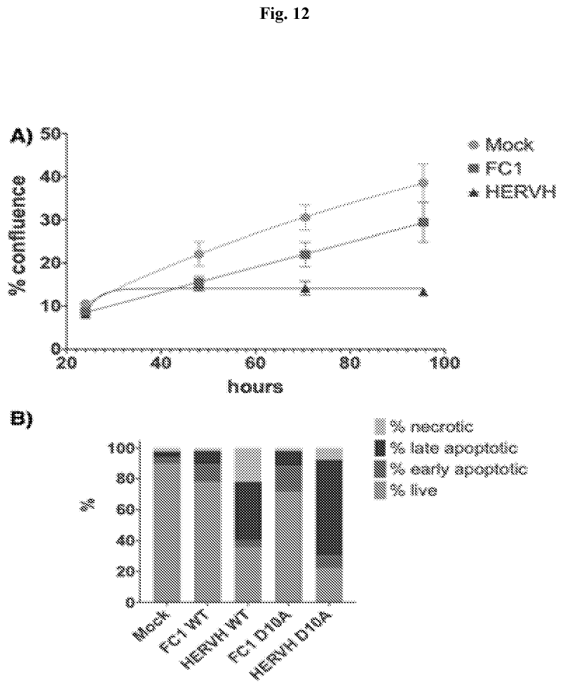

Nonetheless, SpyCas9 D10A nickase or the SpyCas9 nuclease complexed with a sgRNA targeting a sequence in a subset of the HERV-H family was highly toxic in a transformed cell line. See, FIGS. 12A and 12B. In contrast, SpyCas9 D10A nickase or the SpyCas9 nuclease complexed with an sgRNA targeting an LTR sequence in HERV-FC1 (which is an HERV that is present on a single copy on the X chromosome) was not toxic to these cells. Bénit et al., “Characterization of the low-copy HERV-Fc family: evidence for recent integrations in primates of elements with coding envelope genes” Virology 312:159-168 (2003). Similar toxicity has been reported when employing cytosine base editors to inactivate porcine endogenous retroviruses (PERVs), which nick the DNA and deaminate cytosine. Smith et al., “Enabling large-scale genome editing at repetitive elements by reducing DNA nicking” Nucleic Acids Res 48:5183-5195 (2020).

Although it is not necessary to understand the mechanism of an invention, it is believed that genomic nick-induced cell toxicity would also be experienced by cancer cells containing genomic amplifications when these regions are targeted by a nickase. It has been reported that cancer cells can amplify hundreds of copies of a genomic region, which would be similar in number to the HERV-H target sites that produced cellular toxicity when targeted by a nickase. Zack et al., “Pan-cancer patterns of somatic copy number alteration” Nat Genet 45:1134-1140 (2013); and Beroukhim et al., “The landscape of somatic copy-number alteration across human cancers” Nature 463:899-905 (2010). It is further believed that limited cell toxicity will results in non-cancerous cells treated with a nickase targeting a genome amplified region in cancer cells since these genome regions are not amplified in normal cells.

Neuroblastoma cell lines commonly have genomic amplifications in an MycN gene. Reiter et al., “MYCN is the only highly expressed gene from the core amplified domain in human neuroblastomas” Genes Chromosomes Cancer 23:134-140 (1998); Mathew et al., “Detection of MYCN Gene Amplification in Neuroblastoma by Fluorescence In Situ Hybridization: A Pediatric Oncology Group Study” Neoplasia 3:105-109 (2001); and Roy et al., “Combined M-FISH and CGH analysis allows comprehensive description of genetic alterations in neuroblastoma cell lines” Genes Chromosomes Cancer 32:126-135 (2001).

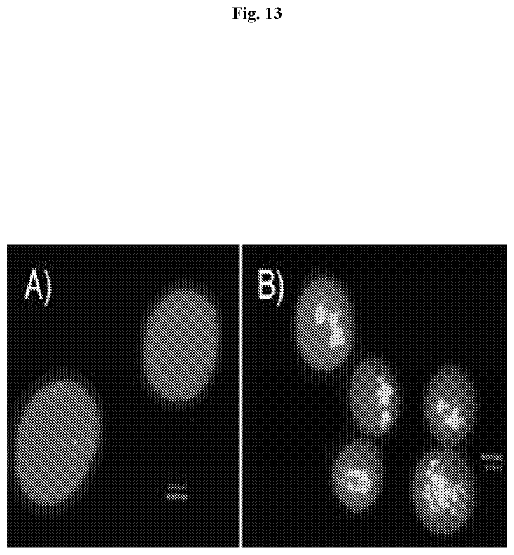

The impact of Cas9 nuclease and Cas9 nickase activity was examined with two different neuroblastoma cell lines: i) SY5Y, which do not have MycN amplifications; and ii) Kelly, which have MycN amplifications that can exceed 1000 copies. See, FIG. 13. SpyCas9 nuclease or D10A Cas9 nickase proteins were complexed to different synthetic sgRNAs (i.e., a ribonucleotide protein complex; RNP) and delivered to these cells by electroporation and then cell proliferation was measured (Celigo Imaging Cytometer).

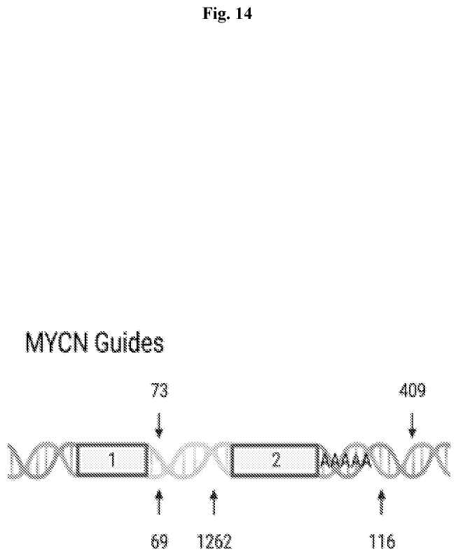

Three different target sites were used to assess cell toxicity: two sgRNAs targeting different positions within the first intron of MycN (MycN 69 and MycN 1262; and a sgRNA targeting HERV-FC1. See, FIG. 14; and Table 2.

TABLE 2
Representative guide RNA positions For HIV, HERV and MYCN genomic regions

| Guide | Locus | Sequence (5′-3′) | hg38 coordinates |
|---|---|---|---|
| FC1 358 | HERV-Fc1 | TGTCTTCCCAAGCCGGACAT | chrX:97841839-97841858 |
| HERVH 2 | HERV-H | AGGTCCCCCGATCCGAGTCA | chrX:92079892-92079911* |
| MYCN int 69 | MYCN | GCACGTCGCAATCCCGTCCA | chr2:16080934-16080953 |
| MYCN int 73 | MYCN | CGGGATTGCGACGTGCGCAC | chr2:16080938-16080957 |
| MYCN outside 116 | MYCN | CCCCCCAACCAGGATTGTAC | chr2:16087244-16087263 |
| MYCN outside 409 | MYCN | TTTGGTAGTATTCGTCCCAT | chr2:16087514-16087533 |
| MYCN int 1262 | MYCN | CCCGTTCGTTTTAATACCGG | chr2:16082127-16082146 |
| HIV LTR 485 | HIV | GGGAGCTCTCTGGCTAACTA | n/a |
| HIV LTR 600 | HIV | ACACTGACTAAAAGGGTCTG | n/a |
| HIV LTR 616 | HIV | GTGTGGAAAATCTCTAGCAG | n/a |
| HIV LTR 628 | HIV | TCTAGCAGTGGCGCCCGAAC | n/a |
| HIV LTR 644 | HIV | TCGCTTTCAAGTCCCTGTTC | n/a |

*Example locus-there are 179 cognate sites in the genome

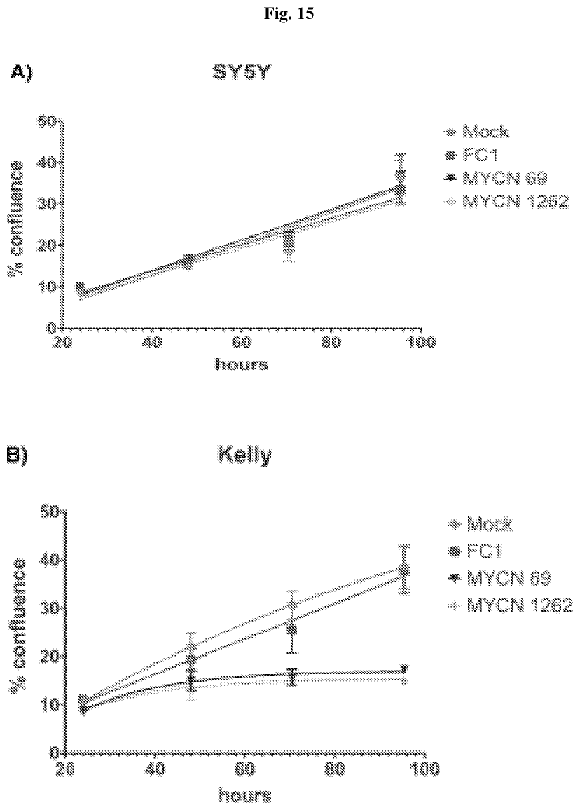

As shown above, HERV-FC1 can undergo efficient genomic collapse to a single LTR by targeting with SpyCas9 D10A nickase. Consistent with this observation, when treating each cancer cell line with SpyCas9 nuclease RNPs, cell toxicity was only observed in the Kelly cells when Cas9 was programmed for either target site in MycN. See, FIGS. 15A and 15B. Cell toxicity was observed in Kelly cells but not SY5Y cells when targeting MycN, which is consistent with other reports of cell toxicity when targeting amplified genomic regions with nucleases. Wang et al., “Identification and characterization of essential genes in the human genome” Science 350:1096-1101 (2015).

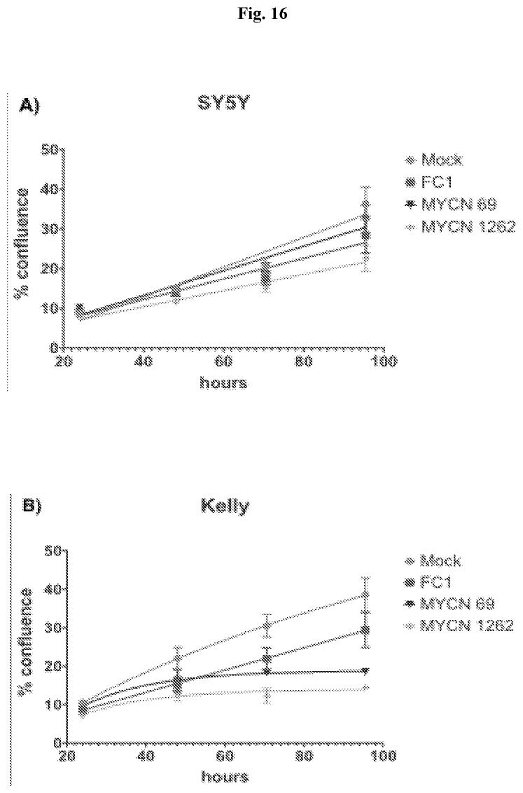

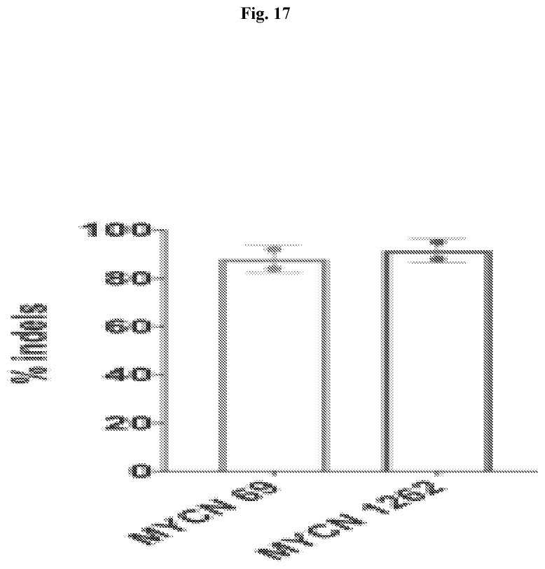

When treating each cell line with SpyCas9 D10A nickase RNPs, cell toxicity was again only observed in the Kelly cells when Cas9 was programmed for either target site in MycN. See, FIGS. 16A and 16B. The decrease in cell proliferation rates (e.g., cell toxicity) for the population of Kelly cells treated with the SpyCas9 nuclease and the D10A nickase were similar when targeting the same target sites. The absence of cell toxicity for the MycN sgRNAs in SY5Y cells is not due to the absence of genome editing, as the SpyCas9 nuclease produces high levels of mutations (>80% insertions and deletions; InDels) at each target site determined by a TIDE analysis. See, FIG. 17; and Brinkman et al., “Easy quantitative assessment of genome editing by sequence trace decomposition” Nucleic Acids Res 42, e168 e168 (2014).

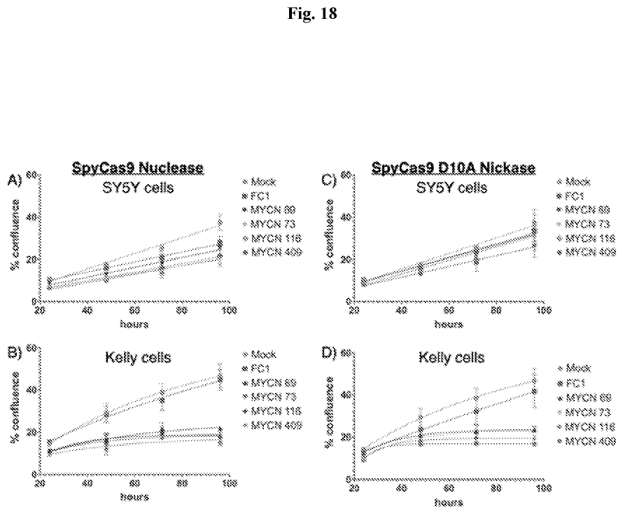

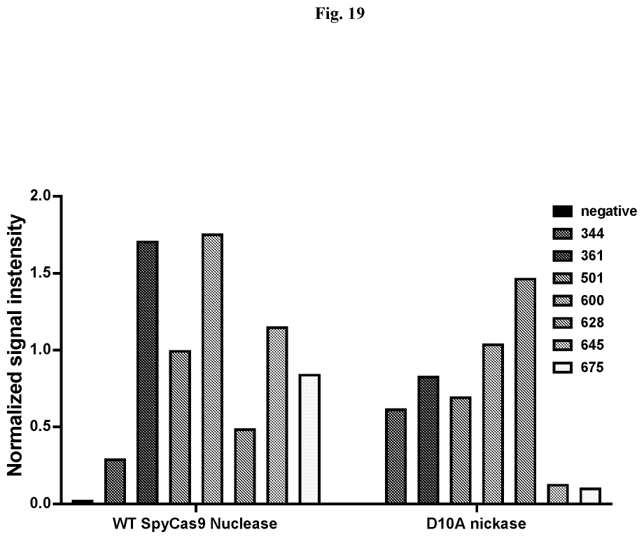

The toxicity of the SpyCas9 D10A nickase is not limited to sgRNAs that target the first intron of MycN. Other guide RNAs targeting downstream of the transcriptional unit also have similar impacts of the proliferation of Kelly cells, but little impact on the growth of SY5Y cells that lack the amplified region. See, FIGS. 18A-D. Thus, these data show that five (5) different guide RNAs targeting the MycN locus can yield substantial impact on cell viability. Overall these data demonstrate that SpyCas9 D10A nickases, like nucleases, can be highly toxic to cell proliferation when targeting an amplified genomic region.

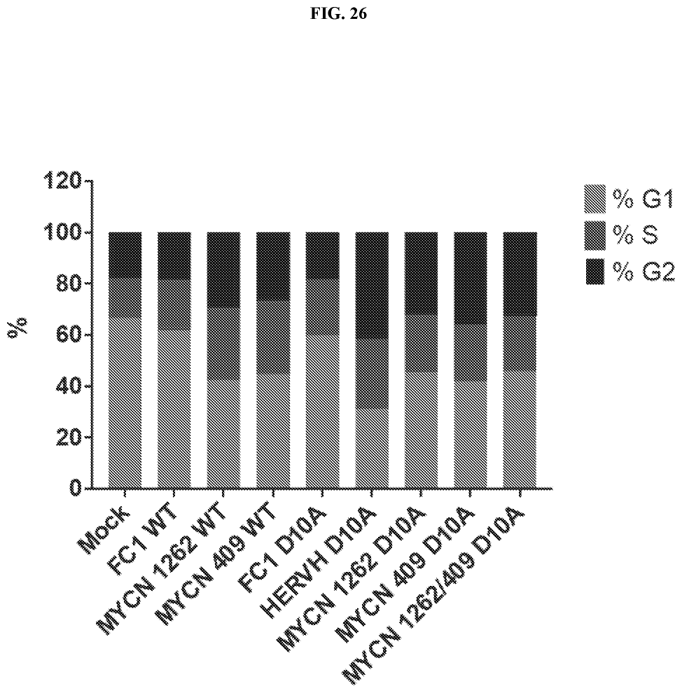

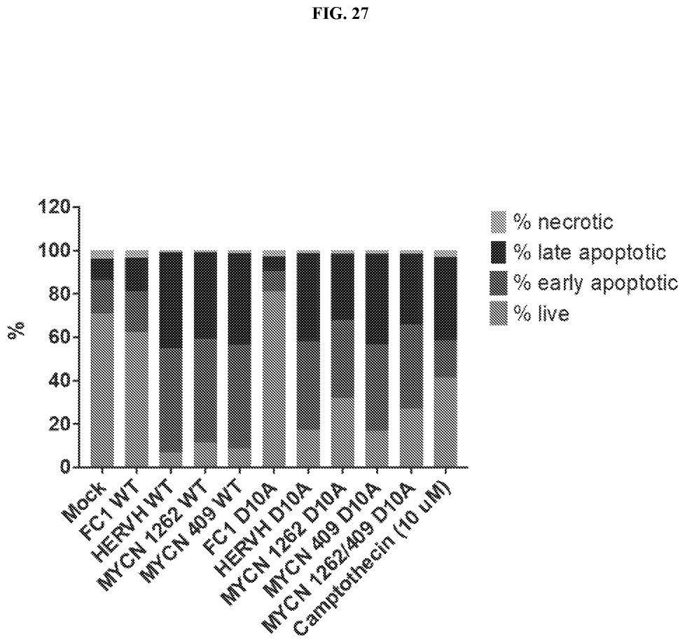

MycN amplification toxicity was also assessed using Kelly cells containing an MycN amplification were nucleofected with the indicated Cas9 nuclease (WT) RNP or D10ACas9 nickase (D10A) programmed with the indicated guideRNA(s). After 72 hours, these Kelly cells were Hoechst stained to determine the fraction of cells that are in each phase of the cell cycle. See, FIG. 26. Cas9 Nuclease (WT) or D10A nickase (D10A) RNP composition in each sample is indicated on the X-axis. HERV FC1 is likely a single target in the genome. HERVH and MYCN have numerous copies in these cells and are sensitive to nicks or DSBs, where more cells are stuck in the S and G2 phases of the cell cycle. Camptothecin treatment is included to provide a reference for non-specific replication fork collapse and DSB introduction. After 72 hours, these Kelly cells were also PI/AnnexinV stained to test for viability and the fraction of cells that were undergoing apoptosis is indicated. See, FIG. 27. Cas9 Nuclease (WT) or D10A nickase (D10A) RNP composition in each sample is indicated on the X-axis. HERV FC1 is likely a single target in the genome. HERVH and MYCN have numerous copies in these cells and are sensitive to nicks or DSBs, which is producing high rates of apoptosis. Camptothecin treatment is included to provide a reference for non-specific replication fork collapse and DSB Introduction

Although all of the experimental work to date has focused on SpyCas9 nickase, any programmable nickase with good activity and specificity should be applicable for this approach. Other Cas9 orthologs nickases, Cas9 sequence variant nickases, zinc finger nickases (where one of the two FokI domains are functional), transcription activator like effector nickases (where one of the two FokI domains are functional), Cas12a nickases, CasX nickases, meganuclease nickases, megaTAL nickases should all be applicable to this approach.

### IV. Pharmaceutical Compositions

The present invention further provides pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides or proteins, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### V. Drug Delivery Systems

The present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids.

Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease*, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA PGA Copolymer Ratios, J. Biomed. Mater. Res., Vol. I1:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 m and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well-known material.

We claim:

1. A method, comprising:
a) providing;
i) a Cas9 nickase protein;
ii) a deoxyribonucleic acid (DNA) comprising a plurality of repeat regions; and
iii) a guide ribonucleic acid (gRNA) having a sequence complementary to a target site proximate to, or overlapping with, at least one of said plurality of repeat regions;
b) complexing said Cas9 nickase with said gRNA;
c) hybridizing said gRNA to said target site such that said Cas9 nickase creates a nick proximate to, or overlapping with, at least one of said plurality of repeat regions; and
d) deleting said at least one of said plurality of repeat regions from said DNA.

2. The method of claim 1, wherein said method further comprises repeating steps (b) through (d).

3. The method of claim 1, wherein said deleting comprises a genomic collapse.

4. The method of claim 1, wherein said DNA comprises at least one retrovirus.

5. The method of claim 4, wherein said at least one retrovirus is a porcine endogenous retrovirus.

6. The method of claim 4, wherein said at least one retrovirus is a pathogenic human retrovirus.

7. The method of claim 1, wherein said DNA is a genomic DNA.

8. The method of claim 7, wherein said genomic DNA is from a cancer cell.

9. The method of claim 8, wherein said cancer cell is a human cancer cell.

10. The method of claim 7, the genomic DNA is from a T-cell.

11. The method of claim 10, wherein said T-cell is a human T cell.

12. The method of claim 7, wherein said genomic DNA is a human genomic DNA.

13. The method of claim 7, wherein said genomic DNA is a porcine genomic DNA.

14. The method of claim 1, wherein said method does not induce a mutation in said DNA.

15. A method, comprising:
a) providing;
i) a Cas9 nickase protein;
ii) a porcine cell comprising a deoxyribonucleic acid (DNA) having a plurality of endogenous retroviral repeats; and
iii) a guide ribonucleic acid (gRNA) having a sequence complementary to a target site proximate to, or overlapping with, at least one of said plurality of endogenous retroviral repeats;
b) administering said Cas9 nickase and said gRNA to said porcine cell such that said Cas9 nickase creates a nick proximate to, or overlapping with, at least one of said plurality of endogenous retroviral repeats; and
c) deleting said at least one of said plurality of endogenous retroviral repeats from said porcine DNA.

16. The method of claim 15, wherein said method further comprises repeating steps (b) and (c) thereby creating an endogenous retroviral repeat-free porcine.

17. The method of claim 15, wherein said method does not induce a mutation in said porcine DNA.

18. A method, comprising:
a) providing;
i) a Cas9 nickase protein;
ii) a patient comprising a cancer cell having a deoxyribonucleic acid (DNA) with a plurality of amplified genomic regions; and
iii) a guide ribonucleic acid (gRNA) having a sequence complementary to a target site proximate to, or overlapping with, at least one of said plurality of amplified genomic regions;
b) administering said Cas9 nickase and said gRNA to said patient such that said Cas9 nickase creates a nick proximate to at least one of said plurality of amplified genomic regions; and
c) deleting or destabilizing said at least one of said plurality of amplified genomic regions from said patient DNA.

19. The method of claim 18, wherein said method further comprises repeating steps (b) and (c) thereby selectively killing the cancer cell.

20. The method of claim 18, wherein said method is not toxic to a non-cancer cell of said patient.

21. The method of claim 18, wherein said method does not induce a mutation in a non-cancer cell DNA of said patient.

22. The method of claim 18, wherein said patient is a human patient.

23. The method of claim 18, wherein said method selectively kills the cancer cells.

24. The method of claim 18, wherein said method does not comprise repeating steps (b) and (c) and said method selectively kills the cancer cells.
